(19) United States
(12) Patent Application Publication
(10) Pub. No.: US 2021/0403568 A1
(43) Pub. Date: Dec. 30, 2021

(54) BIOPHARMACEUTICAL FORMULATION OF ANTI-PD-1, ANTI-PD-L1, AND ANTI-VEGFR THERAPEUTIC MONOCLONAL ANTIBODIES AND METHOD FOR TREATING NSCLC BY INHALATION

(71) Applicants: Cai Gu Huang, Shrewsbury, MA (US); Hitesh Bhagavanbhai Mangukiya, Shanghai (CN)

(72) Inventors: Cai Gu Huang, Shrewsbury, MA (US); Hitesh Bhagavanbhai Mangukiya, Shanghai (CN)

(73) Assignee: Cai Gu Huang, Shrewsbury, MA (US)

(21) Appl. No.: 17/362,218

(22) Filed: Jun. 29, 2021

Related U.S. Application Data
(60) Provisional application No. 63/045,759, filed on Jun. 29, 2020.

(51) Int. Cl. C07K 16/28; A61K 9/00; C07K 16/22; A61K 47/22; A61K 47/26; A61K 47/12; A61K 47/02; A61K 39/395

(52) U.S. Cl. CPC C07K 16/2818; A61K 9/0078; C07K 16/2827; C07K 16/22; A61K 2039/544; A61K 47/26; A61K 47/12; A61K 47/02; A61K 39/39591; A61K 47/22

(57) ABSTRACT

This invention relates to pharmaceutical formulations of therapeutic monoclonal antibody drugs and pharmaceutically acceptable excipients and a novel therapeutic strategy for the treatment of lung cancers including metastatic NSCLC by administration of such formulations using a soft mist inhaler and/or nebulizer. The pharmaceutical formulations comprise (a) a therapeutic monoclonal antibody selected from the group consisting of pembrolizumab, atezolizumab, nivolumab, durvalumab, and bevacizumab, (b) water, and (c) a buffer. The pharmaceutical formulations are delivered locally to the lungs by inhalation for treatment of cancer.

### PRIORITY STATEMENT

This application claims the benefit of the filing date of U.S. Provisional Patent Application No. 63/045,759, filed on Jun. 29, 2020, which is incorporated herein by reference in its entirety.

### BACKGROUND OF THE INVENTION

More therapeutic monoclonal antibodies and antibody-based modalities are in development today than ever before, and a faster and more accurate drug discovery process will ensure that the number of candidates coming to the biopharmaceutical pipeline will increase in the future.

Cancer is one of the leading causes of death worldwide. Lung cancer in particular, is among the top 3 most prevalent cancers and has a very poor survival rate. (Five-year relative survival rate is about 6% as per the Surveillance, Epidemiology, and End Results database.) Despite the availability of many cancer drugs it has been difficult and, in the case of some cancer types, almost impossible to improve cure rates or survival. There are many reasons for this lack of success but one reason is the inability to deliver adequate amounts of the drugs to the tumor without causing debilitating and life threatening toxicities in the patient. Indeed, most chemotherapeutic drugs used to treat cancer are highly toxic to both normal and tumor tissues.

Important advancements in the treatment of non-small cell lung cancer (NSCLC) have been achieved over the past two decades, increasing our understanding of the disease biology and mechanisms of tumor progression, and advancing early detection and multimodal treatment. The use of small molecule tyrosine kinase inhibitors and immunotherapy has led to unprecedented survival benefits in selected patients. However, the overall cure and survival rates for NSCLC remain low, particularly in metastatic disease. Medications can be taken in a variety of ways like by swallowing, by inhalation, by absorption through the skin, or by intravenous injection. Each method has advantages and disadvantages, and not all methods can be used for every medication. Improving current delivery methods or designing new ones can enhance the efficacy and use of existing medications to expand the clinical benefit to a broader patient population and to improve outcomes in NSCLC.

As part of the Biologics Price Competition and Innovation Act (BPCIA), a biological drug product (produced in or derived from living organisms) may be demonstrated to be “biosimilar” if data show that, among other things, the product is “highly similar” to an already-approved biological product. The biosimilar product should retain at least the biologic function and treatment efficacy of the U.S. Food and Drug Agency-approved biological product. The biological product can be formulated differently, however, from the approved biological product. The formulation can improve stability and shelf storage of the biologic drug product, and can also improve the efficacy in treating a particular disease or condition. The formulation can also improve other aspects of administration, including a reduction in patient discomfort or other unwanted effects that a patient may experience upon administration of the approved biological product. Antibody molecules can be produced as a biosimilar and reformulated accordingly. There remains a need in the art for high quality antibody formulations, methods of administration, and uses thereof.

Currently, the systemic intravenous administration of a lung cancer drug can only deliver about 9-10% of the drug at a tumor site in the lung, which means that a high dose of cancer medicine is generally required. In addition, administration by the intravenous route exposes the entire body to the drug. Doses are selected that destroy tumor cells, but these doses also destroy normal cells. As a result, the patient usually experiences severe toxic side effects. For example, severe myelosuppression may result, which compromises the ability of the patient to resist infection and allows spread of the tumor. There are other life-threatening effects such as hepatotoxicity, renal toxicity, pulmonary toxicity, cardiotoxicity, neurotoxicity, and gastrointestinal toxicity caused by anticancer drugs. Moreover, a significant amount of drug remains in the circulatory system and causes severe side effects as well as other adverse effects. These toxicities are not present to the same extent with all anticancer drugs but are all due to systemic delivery of the drug.

The differences in mechanisms of action and pharmacokinetic properties determine, in part, the efficacy of the various anticancer drugs against different tumor types, which exhibit various biological behaviors.

Local drug delivery by inhalation is proposed as a method for delivering high drug concentrations to a target site while preventing exposure of vital organs to toxic drug concentrations in the systemic circulation. In this way, systemic side effects are minimized. The respiratory system has a large surface area, thin alveolar epithelium, rapid absorption, lack of first-pass metabolism, high bioavailability, and the capacity to absorb large quantities of drug, making it an optimal route of drug administration (Labiris and Dolovich 2003).

It is clinically advantageous to use a liquid formulation of therapeutic monoclonal antibody drug administered using suitable inhalers to achieve localized delivery of the active substances into the lung. Drug delivery to the lungs can be improved by increasing the lung deposition of the effective cancer medicine. Moreover, lung deposition of the drug delivered by inhalation can be increased by administering the drug using soft mist inhalation or nebulize inhalation. A soft mist inhalation device or other nebulization devices can significantly increase the lung deposition and therefore drug delivery of liquid drug formulations.

In U.S. Pat. No. 6,471,943 B 1 to Michael E. Placke, the patent disclosure suggests that highly toxic vesicant and previously unknown nonvesicant antineoplastic drugs can be effectively delivered to a patient in need of treatment for neoplasms or cancers by inhalation. This route is particularly effective for treatment of neoplasms or cancers of the pulmonary system because the highly toxic drugs are delivered directly to the site where they are needed, providing regional doses much higher than can be achieved by conventional IV delivery. As used herein, the respiratory tract includes the oral and nasal-pharyngeal, tracheobronchial, and pulmonary regions. The pulmonary region is defined to include the upper and lower bronchi, bronchioles, terminal bronchioles, respiratory bronchioles, and alveoli.

Cancer immunotherapy has traditionally involved complicated methods using cells and individualized and time-consuming preparations. Recently, monoclonal antibody-based cancer immunotherapy based on the interruption of suppressive signals that are delivered to the adaptive immune system has shown promise in the clinic within the setting of off-the-shelf systemic immunotherapy. However, there is a continuing need in the art to obtain safer and more effective treatments for cancer.

Konstantinos Sapalidis et. al. recently studied the three immunotherapeutic drugs nivolumab, ipilimumab and pembrolizumab that can be produced as aerosols with water as a solvent using a jet-nebulizer and residual cup (Sapalidis, Zarogoulidis et al. 2018).

The U.S. FDA has approved the following recently-developed therapeutic monoclonal antibodies only for intravenous administration for metastatic NSCLC. Pembrolizumab, as a single agent, is indicated for the first-line treatment of patients with metastatic NSCLC whose tumors have high PD-L1 expression (Tumor Proportion Score (TPS) ≥50%) as determined by an FDA-approved test, with no EGFR or ALK genomic tumor aberrations and/or with disease progression on or after platinum-containing chemotherapy. Atezolizumab and nivolumab are separately indicated for the treatment of patients with metastatic NSCLC who have disease progression during or following platinum-containing chemotherapy patients with EGFR or ALK genomic tumor aberrations should have disease progression on FDA-approved therapy for these aberrations prior to receiving atezolizumab or nivolumab. Durvalumab is indicated for the treatment of patients with unresectable stage III SCLC whose disease has not progressed following concurrent platinum-based chemotherapy and radiation therapy. Bevacizumab in combination with carboplatin and paclitaxel, is indicated for the first-line treatment of patients with unresectable, locally advanced, recurrent or metastatic NSCLC.

Objectives in formulating a therapeutic monoclonal antibody solution for administration by inhalation include increasing the efficacy of the therapeutic monoclonal antibody, and reducing the dosage and side effects compared with those from intravenous infusion treatment of NSCLC. In general, disadvantages of administration of therapeutic monoclonal antibodies by intravenous infusion include the route of administration, the high doses required, and the stability of the formulations; once an infusion is prepared it has to be administered through an intravenous line as soon as possible, or it can be stored only for 24 hours in a refrigerator at 2° C. to 8° C. (36° F. to 46° F.) or 8 hours at room temperature.

### SUMMARY OF THE INVENTION

The present invention comprises formulations of therapeutic monoclonal antibody drugs and pharmaceutically acceptable excipients and a novel therapeutic strategy for the treatment of metastatic NSCLC by administration of such formulations using a soft mist inhaler and/or nebulizer. The biopharmaceutical formulations according to the invention meet high quality standards.

One aspect of the present invention is to provide a biopharmaceutical formulation containing one or more cancer therapeutic monoclonal antibodies and optional excipients, which meets high quality standards and is able to achieve optimum nebulization of a solution administered using a soft mist inhaler. In an embodiment, the pharmaceutical formulations of the invention are stable and can be stored for at least about a few months to a few years, such as about one year, or such as about two years. In one embodiment, the storage temperature is less than about 4° C.

Another aspect of the invention is to provide biopharmaceutical formulations of nebulization solutions comprising one or more anti-cancer therapeutic monoclonal antibodies which formulations are nebulized by inhalation devices. In an embodiment, the nebulized formulation produces an aerosol with a droplet size falling reproducibly within a specified range. In one embodiment, the average particle size is less than about 10 microns.

Another aspect of the present invention is to provide biopharmaceutical solution formulations comprising one or more therapeutic monoclonal antibodies and optional excipients which can be administered by nebulization using ultra-sonic-based or air pressure-based nebulizers/inhalers. In an embodiment, the pharmaceutical formulations of the invention are stable and can be stored for at least about a few months to a few years, such as about one year, or such as about two years. In one embodiment, the storage temperature is less than about 4° C.

In an embodiment, the current invention provides stable biopharmaceutical formulations containing one or more therapeutic monoclonal antibodies (such as anti-PD-1, anti-PD-L1, and anti-VEGFR) and optional excipients useful for treating metastatic NSCLC. In an embodiment, the pharmaceutical formulations of the invention can be delivered in the form of an aerosol by soft mist inhalation produced by atomizer inhalation devices. In an embodiment, the aerosolized therapeutic monoclonal antibodies produced according to the invention are locally delivered to a lung tumor by inhalation. Local delivery of a therapeutic monoclonal antibody may increase the efficacy in treating metastatic NSCLC by increasing lung deposition of the therapeutic monoclonal antibody. This therapeutic strategy may reduce side effects of the drug because only low concentrations of the antibody will be absorbed through the alveoli and reach the circulatory system. Local delivery of a therapeutic monoclonal antibody by inhalation may also reduce the dose of the therapeutic antibody required compared to the dose for systematic intravenous administration, which may lead to reduced toxicity.

### BRIEF DESCRIPTION OF THE DRAWINGS

FIG. 1 shows a longitudinal section through an inhalation atomizer in the stressed state.

FIG. 2 shows a counter element of an inhalation atomizer.

### DETAILED DESCRIPTION OF THE INVENTION

The technical and nontechnical terminology used herein is for the purpose of describing particular embodiments only and is not intended to limit the invention. Unless otherwise defined, all terms (including technical and scientific terms) used herein have the same meaning as commonly understood by one having ordinary skill in the art to which invention belongs. It will be further understood that terms, such as those defined in commonly used dictionaries, should be interpreted as having a meaning that is consistent with their meaning in the context of relevant art and the present disclosure and will not be interpreted in an idealized or overly formal sense unless expressly so defined herein.

In describing the invention, it will be understood that a number of formulations and steps are disclosed. Each of these has individual benefits and each can also be used in conjugation with one or more, or in some cases all, of the other disclosed techniques. Accordingly, for the sake of clarity, this description will refrain from repeating every possible combination of the individual steps in an unnecessary fashion. Nevertheless, the specification and claims should be read with understanding that such combinations are entirely within the scope of invention and the claims.

The present disclosure is to be considered as an exemplification of the invention, and is not intended to limit the invention to the specific embodiments illustrated by the figures or description below.

One aspect of the present invention is to achieve better and significant delivery of therapeutic biologics to the lung for treatment of metastatic NSCLC. Another aspect of the present invention is to increase the lung deposition of a drug delivered by inhalation method. In yet another aspect of the invention, inhalation drug delivery is improved by increasing deposition of the drug in the lungs. The soft mist or nebulization inhalation device disclosed in US20190030268 can significantly increase the lung deposition of inhalable drugs. Such soft mist or nebulization inhalation devices can nebulize a small amount of a liquid formulation within a few seconds into an aerosol that is suitable for therapeutic inhalation. Such soft mist or nebulization inhalation devices are particularly suitable for administering the liquid formulations of the present invention.

Soft mist devices suitable for administering the biopharmaceutical formulations of the present invention are those in which an amount of less than about 70 microliters of pharmaceutical solution can be nebulized in one puff, such as less than about 30 microliters, or less than about 15 microliters, so that the inhalable part of aerosol corresponds to the therapeutically effective quantity of drug. An average particle size of aerosol formed from one puff is less than about 15 microns, or less than about 10 microns.

Nebulization devices suitable for administering the biopharmaceutical formulations of the present invention are those in which an amount of less than about 8 milliliters of biopharmaceutical solution can be nebulized in one puff, such as less than about 2 milliliters, or less than about 1 milliliter, so that the inhalable part of aerosol corresponds to the therapeutically effective quantity. An average particle size of aerosol formed from one puff is less than about 15 microns, or less than about 10 microns.

A suitable device for the propellant-free administration of a metered amount of a liquid pharmaceutical composition for inhalation according to the invention is described in detail, for example, in US20190030268 “inhalation atomizer comprising a blocking function and a counter”.

In an embodiment, the pharmaceutical formulation in the nebulizer is converted into an aerosol destined for the lungs. In an embodiment, the pharmaceutical formulation is sprayed by the nebulizer using high pressure.

In an embodiment, the pharmaceutical formulation is stored in a reservoir in an inhalation device. The pharmaceutical formulation of solutions does not contain any ingredients which might interact with the inhalation device to affect the pharmaceutical quality of the solution or of the aerosol produced. In addition, in an embodiment, the active substances in the pharmaceutical formulations according to the present invention are very stable when stored and can be administered directly.

Therefore, one aspect of the present invention is to provide a biopharmaceutical formulation containing a therapeutic monoclonal antibody and optional excipients, which meets high standards to achieve optimum nebulization for administration using a soft mist inhaler or nebulizer. In an embodiment, the pharmaceutical formulations of the invention are stable and can be stored for at least about a few years, such as about one year, or such as about three years. In one embodiment, the storage temperature is less than about 4° C.

In an embodiment, the present invention is a biopharmaceutical formulation of a therapeutic monoclonal antibody as an active substance with optional excipients in a solution, which can be administered by soft mist inhalation or nebulization inhalation.

In an embodiment, the current invention provides a method for the treatment of metastatic NSCLC or other type of lung diseases and human cancers. In an embodiment, the biopharmaceutical formulations of the present invention for administration by soft mist inhaler and/or nebulizer meet standard quality guidelines. One aspect of the current invention is to provide a stable formulation containing at least one therapeutic monoclonal antibody in functional form with inactive ingredients, which meets the standard delivered dosage requirement for optimum nebulization of a solution using a soft mist inhaler and/or nebulizer. In an embodiment, the pharmaceutical formulation is formulated in a stable solution to maintain the active ingredient functionality at the labeled dosage. In another aspect, the present invention provides a propellant-free suspension containing at least one therapeutic monoclonal antibody and excipients, which is nebulized under pressure using a soft mist or nebulization inhalation device. In an embodiment, the pharmaceutical formulation produces an aerosol with a droplet size falling reproducibly within a specified range. In one embodiment, the average particle size of the aerosol droplets is less than about 10 microns in diameter.

In another aspect, the invention provides a method for treating a condition in a subject comprising administering to the subject in need thereof an effective amount of the pharmaceutical composition as described herein. In some embodiments, the condition is a cancer. In some embodiments, the cancer is selected from the group consisting of lung cancer, gastric, sarcoma, lymphoma, leukemia, head and neck cancer, thymic cancer, epithelial cancer, salivary cancer, liver cancer, stomach cancer, thyroid cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma. In some embodiments, the cancer is a platinum resistant and/or platinum refractory cancer, such as, for example, platinum resistant and/or refractory lung cancer, platinum resistant and/or refractory breast cancer, or platinum resistant and/or refractory ovarian cancer.

In another aspect, the invention relates to a method of inhibiting tumor growth or progression in a subject who has a tumor, comprising administering to the subject an effective amount of the pharmaceutical composition by soft mist inhaler of nebulizer as described herein.

In another aspect, the invention relates to a method of inducing tumor regression in a subject who has a PD-1 expressing tumor, comprising administering to the subject an effective amount of the pharmaceutical composition by soft mist inhaler or nebulizer as described herein.

In another aspect, the invention relates to a method of inducing tumor regression in a subject who has a PD-L1 expressing tumor, comprising administering to the subject an effective amount of the pharmaceutical composition by soft mist inhaler or nebulizer as described herein.

In another aspect, the invention relates to a method of inducing tumor regression in a subject who has a VEGFR expressing tumor, comprising administering to the subject an effective amount of the pharmaceutical composition by soft mist inhaler or nebulizer as described herein.

In another aspect, the present disclosure provides a method for enhancing the immunogenicity or therapeutic effect of a vaccine delivered by soft mist inhalation or nebulization for the treatment of a cancer in a mammal, particularly a human, which method comprises administering to the mammal receiving the vaccine an effective amount of antibodies provided by the present disclosure.

In one embodiment, the antibody is a full-length antibody, comprising both variable and constant regions, although in some embodiments, the antibody may comprise a derivative or fragment or portion of a full-length antibody that retains the antigen-binding specificity, and also may retain most or all of the affinity, of the full length antibody molecule. The antibody may comprise post-translational modifications (PTMs) or moieties, which may impact antibody activity or stability. The antibody may be methylated, acetylated, glycosylated, sulfated, phosphorylated, carboxylated, and/or amidated, and may comprise other moieties that are well known in the art. In an embodiment, the formulation comprises a therapeutically effective amount of the antibody.

A therapeutically effective amount may vary, depending on the disease or condition being treated upon administration of the antibody, and/or depending on the characteristics of the subject to which the antibody is administered, such as age, gender, height, weight, state of advancement or stage of the disease or condition, the number and efficacy of previous administrations, other therapeutic agents administered to the subject, and other characteristics that are known to the practitioner or that would otherwise be taken into account in determining appropriate dosing. In one embodiment, a therapeutically effective amount is an amount that is effective to treat cancers such as non-squamous non-small cell lung cancer, glioblastoma, renal cell carcinoma, cervical cancer, or epithelial ovarian, breast cancer, fallopian tube, or primary peritoneal cancer. For systemic administration, the daily dose of the antibody ranges from about 1 μg to about 2000 mg per dose. In one embodiment, the daily dose of the antibody ranges from about 10 mg to about 800 mg per dose. In one embodiment, the daily dose of the antibody ranges from about 10 mg to about 200 mg per dose. In one embodiment, the daily dose of the antibody ranges from about 200 mg to about 600 mg per dose. In one embodiment, the daily dose of the antibody ranges from about 10 μg to about 200 μg per dose. In one embodiment, the daily dose of the antibody ranges from about 10 μg to about 50 μg per dose.

The formulation may comprise from about 1 mg/ml to about 100 mg/ml of the antibody. In some aspects, the formulation comprises from about 5 mg/ml to about 40 mg/ml of the antibody. In some aspects, the formulation comprises from about 30 mg/ml to about 50 mg/ml of the antibody. In some aspects, the formulation comprises from about 20 mg/ml to about 50 mg/ml of the antibody. In some aspects, the formulation comprises from about 20 mg/ml to about 40 mg/ml of the antibody. In some aspects, the formulation comprises from about 4 mg/ml to about 12 mg/ml of the antibody. In some aspects, the formulation comprises from about 10 mg/ml to about 55 mg/ml of the antibody. In some aspects, the formulation comprises from about 5 mg/ml to about 30 mg/ml of the antibody. In some aspects, the formulation comprises from about 2 mg/ml to about 8 mg/ml of the antibody. In some aspects, the formulation comprises from about 55 mg/ml to about 65 mg/ml of the antibody. In some aspects, the formulation comprises from about 15 mg/ml to about 25 mg/ml of the antibody. In some aspects, the formulation comprises from about 5 mg/ml to about 15 mg/ml of the antibody. In some aspects, the formulation comprises from about 10 mg/ml to about 20 mg/ml of the antibody. In some aspects, the formulation comprises from about 25 mg/ml to about 35 mg/ml of the antibody. In some aspects, the formulation comprises from about 28 mg/ml to about 32 mg/ml of the antibody. In some aspects, the formulation comprises from about 58 mg/ml to about 62 mg/ml of the antibody. In some aspects, the formulation comprises from about 8 mg/ml to about 12 mg/ml of the antibody. In some aspects, the formulation comprises from about 13 mg/ml to about 18 mg/ml of the antibody. In some aspects, the formulation comprises from about 45 mg/ml to about 55 mg/ml of the antibody. In some aspects, the formulation comprises from about 23 mg/ml to about 28 mg/ml of the antibody.

The antibody, for example, at the concentrations described or exemplified herein, is preferably formulated with a buffered aqueous carrier, and the carrier preferably comprises sterile water. The buffered antibody formulation is preferably in liquid form, and more preferably in liquid form suitable for soft mist inhalation or nebulization. In an embodiment, the pharmaceutical formulation is a solution. In another embodiment, the pharmaceutical formulation is a suspension. The amount of water in the formulation may vary in accordance with the desired volume of the infusion. In some aspects, the buffer comprises L-histidine, sucrose, and a mild surfactant such as polysorbate 80, and maintains the antibody formulation at an acidic pH at about 5.5 to about 5.7. In some alternate aspects, the buffer comprises L-histidine, sodium phosphate, trehalose, sucrose, and a mild surfactant such as polysorbate 20, and maintains the antibody formulation at an acidic pH of from about 5.6 to about 5.8. In some aspects, the buffer comprises L-histidine, mannitol, trehalose, sodium citrate, sodium chloride, and a pentetic acid, and maintains the antibody formulation at an acidic pH of from about 5.8 to about 6.0. When stored in the buffered formulation, the antibody is shelf stable under normal storage conditions.

In alternate aspects, the histidine buffer comprises an aqueous combination in a pre-mixed solution of from about 3 mM to about 8 mM of histidine, from about 0.05 to about 0.13 mM polysorbate 80, and from about 80 mM to about 170 mM of sucrose.

In some alternate aspects, the buffer comprises an aqueous pre-mixed solution of from about 90 mM to about 400 mM of L-histidine, from about 1.5 mM to about 7 mM of polysorbate 20, and from about 550 mM to about 2450 mM of sucrose, and from about 65 mM to about 275 mM of glacial acetic acid.

In another aspect, the buffer comprises an aqueous pre-mixed solution of from about 0.05 mM to about 0.16 of polysorbate 80, from about 13 mM to about 28 mM of sodium citrate dihydrate, from about 24 mM to about 50 mM of sodium chloride, from 80 mM to about 165 mM of mannitol, and from about 0.01 mM to about 0.02 mM of pentetic acid.

In another aspect, the buffer comprises an aqueous pre-mixed solution of from about 3 mM to about 13 mM of L-histidine, from about 0.03 mM to about 0.16 mM of polysorbate 80, from about 3 mM to about 13 mM of L-histidine hydrochloride monohydrate, and from about 68 mM to about 275 mM of trehalose dehydrate.

In another aspect, the buffer comprises an aqueous pre-mixed solution of from about 0.16 mM to about 0.33 mM of polysorbate 20, from about 18 mM to about 50 mM of monobasic sodium phosphate, from about 2 mM to about 7 mM of dibasic sodium phosphate, and from about 60 mM to about 160 mM of trehalose dehydrate.

The buffer may comprise from about 1 mM to about 400 mM the L-histidine. In some aspects, the buffer may comprise from about 1 mM to about 100 mM of L-histidine. In some aspects, the buffer may comprise from about 1 mM to about 10 mM of L-histidine. In some aspects, the buffer may comprise from about 1 mM to about 5 mM of L-histidine. In some aspects, the buffer may comprise from about 5 mM to about 10 mM of L-histidine. In some aspects, the buffer may comprise from about 90 mM to about 400 mM of L-histidine. In some aspects, the buffer may comprise from about 90 mM to about 110 mM of L-histidine. In some aspects, the buffer may comprise from about 2 mM to about 5 mM of L-histidine. In some aspects, the buffer may comprise from about 10 mM to about 15 mM of L-histidine. In some aspects, the buffer may comprise from about 8 mM to about 10 mM of L-histidine. In some aspects, the buffer may comprise from about 390 mM to about 400 mM of L-histidine. In some aspects, the buffer may comprise from about 85 mM to about 105 mM of L-histidine. In some aspects, the buffer may comprise from about 5 mM to about 20 mM of L-histidine. In some aspects, the buffer may comprise from about 95 mM to about 105 mM of L-histidine.

The buffer may comprise polysorbate 80 as a surfactant in a formulation at about 0.05 mg/ml to about 0.2 mg/ml. In some aspects, the buffer may comprise from about 0.03 mM to about 0.16 mM of polysorbate 80. In some aspects, the buffer may comprise from about 0.03 mM to about 0.08 mM of polysorbate 80. In some aspects, the buffer may comprise from about 0.1 mM to about 0.16 mM of polysorbate 80. In some aspects, the buffer may comprise from about 0.03 mM to about 0.06 mM of polysorbate 80. In some aspects, the buffer may comprise from about 0.07 mM to about 0.13 mM of polysorbate 80. In some aspects, the buffer may comprise from about 0.11 mM to about 0.16 mM of polysorbate 80.

The buffer may comprise from about 0.1 mM to about 10 mM of the polysorbate 20. In some aspects, the buffer may comprise from about 0.1 mM to about 0.4 mM of polysorbate 20. In some aspects, the buffer may comprise from about 0.1 mM to about 0.2 mM of polysorbate 20. In some aspects, the buffer may comprise from about 0.3 mM to about 0.4 mM of polysorbate 20. In some aspects, the buffer may comprise from about 1 mM to about 7 mM of polysorbate 20. In some aspects, the buffer may comprise from about 1 mM to about 5 mM of polysorbate 20. In some aspects, the buffer may comprise from about 1 mM to about 4 mM of polysorbate 20. In some aspects, the buffer may comprise from about 4 mM to about 7 mM of polysorbate 20. In some aspects, the buffer may comprise from about 5 mM to about 7 mM of polysorbate 20. In some aspects, the buffer may comprise from about 6 mM to about 7 mM of polysorbate 20. In some aspects, the buffer may comprise from about 6.5 mM to about 7 mM of polysorbate 20.

The buffer may comprise from about 50 mM to about 2450 mM of sucrose. In some aspects, the buffer may comprise from about 60 mM to about 170 mM of sucrose. In some aspects, the buffer may comprise from about 500 mM to about 2450 mM of sucrose. In some aspects, the buffer may comprise from about 150 mM to about 170 mM of sucrose. In some aspects, the buffer may comprise from about 500 mM to about 700 mM of sucrose. In some aspects, the buffer may comprise from about 1000 mM to about 2450 mM of sucrose. In some aspects, the buffer may comprise from about 100 mM to about 700 mM of sucrose. In some aspects, the buffer may comprise from about 2350 mM to about 2450 mM of sucrose.

The buffer may comprise from about 2 mM to about 15 mM of L-histidine hydrochloride monohydrate. In some aspects, the buffer may comprise from about 3 mM to about 5 mM of L-histidine hydrochloride monohydrate. In some aspects, the buffer may comprise from about 5 mM to about 15 mM of L-histidine hydrochloride monohydrate. In some aspects, the buffer may comprise from about 10 mM to about 15 mM of L-histidine hydrochloride monohydrate.

The buffer may comprise from about 10 mM to about 30 mM of sodium citrate dihydrate. In some aspects, the buffer may comprise from about 10 mM to about 15 mM of sodium citrate dehydrate. In some aspects, the buffer may comprise from about 25 mM to about 30 mM of sodium citrate dehydrate. In some aspects, the buffer may comprise from about 10 mM to about 20 mM of sodium citrate dehydrate.

The buffer may comprise from about 20 mM to about 55 mM of sodium chloride. In some aspects, the buffer may comprise from about 20 mM to about 30 mM of sodium chloride. In some aspects, the buffer may comprise from about 40 mM to about 55 mM of sodium chloride. In some aspects, the buffer may comprise from about 20 mM to about 50 mM of sodium chloride. In some aspects, the buffer may comprise from about 45 mM to about 55 mM of sodium chloride.

The buffer may comprise from about 10 mM to about 60 mM of monobasic sodium phosphate monohydrate. In some aspects, the buffer may comprise from about 10 mM to about 30 mM of monobasic sodium phosphate monohydrate. In some aspects, the buffer may comprise from about 30 mM to about 60 mM of monobasic sodium phosphate monohydrate. In some aspects, the buffer may comprise from about 20 mM to about 40 mM of monobasic sodium phosphate monohydrate. In some aspects, the buffer may comprise from about 45 mM to about 55 mM of monobasic sodium phosphate monohydrate.

The buffer may comprise from about 1 mM to about 10 mM of dibasic sodium phosphate dehydrate. In some aspects, the buffer may comprise from about 1 mM to about 5 mM of dibasic sodium phosphate dihydrate. In some aspects, the buffer may comprise from about 2 mM to about 5 mM of dibasic sodium phosphate dihydrate. In some aspects, the buffer may comprise from about 5 mM to about 7 mM of dibasic sodium phosphate dihydrate. In some aspects, the buffer may comprise from about 6 mM to about 7 mM of dibasic sodium phosphate dihydrate.

The buffer may comprise from about 50 mM to about 200 mM of mannitol. In some aspects, the buffer may comprise from about 50 mM to about 100 mM of mannitol. In some aspects, the buffer may comprise from about 100 mM to about 200 mM of mannitol. In some aspects, the buffer may comprise from about 150 mM to about 200 mM of mannitol. In some aspects, the buffer may comprise from about 160 mM to about 180 mM of mannitol. In some aspects, the buffer may comprise from about 160 mM to about 170 mM of mannitol. In some aspects, the buffer may comprise from about 70 mM to about 90 mM of mannitol.

The buffer may comprise from about 50 mM to about 300 mM of trehalose dehydrate. In some aspects, the buffer may comprise from about 50 mM to about 100 mM of trehalose dihydrate. In some aspects, the buffer may comprise from about 100 mM to about 200 mM of trehalose dihydrate. In some aspects, the buffer may comprise from about 200 mM to about 300 mM of trehalose dihydrate. In some aspects, the buffer may comprise from about 50 mM to about 80 mM of trehalose dihydrate. In some aspects, the buffer may comprise from about 140 mM to about 170 mM of trehalose dihydrate. In some aspects, the buffer may comprise from about 250 mM to about 300 mM of trehalose dihydrate. In some aspects, the buffer may comprise from about 150 mM to about 280 mM of trehalose dihydrate. In some aspects, the buffer may comprise from about 270 mM to about 280 mM of trehalose dihydrate.

The buffer may comprise from about 50 mM to about 300 mM glacial acetic acid. In some aspects, the buffer may comprise from about 50 mM to about 100 mM of glacial acetic acid. In some aspects, the buffer may comprise from about 100 mM to about 200 mM of glacial acetic acid. In some aspects, the buffer may comprise from about 200 mM to about 300 mM of glacial acetic acid. In some aspects, the buffer may comprise from about 50 mM to about 80 mM of glacial acetic acid. In some aspects, the buffer may comprise from about 250 mM to about 290 mM of glacial acetic acid.

The buffer may comprise from about 0.005 mM to about 0.025 mM pentetic acid. In some aspects, the buffer may comprise from about 0.01 mM to about 0.02 mM of pentetic acid. In some aspects, the buffer may comprise from about 0.005 mM to about 0.015 mM of pentetic acid. In some aspects, the buffer may comprise from about 0.015 mM to about 0.025 mM of pentetic acid.

The pharmaceutical formulations of the current invention are especially suitable for administration by soft mist inhalation or nebulization, which provide better lung depositions, typically up to 55-60%, compared to the drugs' bioavailability at the site of action through intravenous infusion specifically for NSCLC. Furthermore, liquid biologics formulations for administration via inhalation may have advantages compared to the administration of therapeutic monoclonal antibodies by intravenous administration, particularly for treatment of NSCLC.

Soft mist inhalers that nebulize a small amount of a liquid formulation containing the required dosage of a therapeutic monoclonal antibody within a few seconds into an aerosol are suitable for therapeutic inhalation. Such soft mist inhalers are particularly suitable to the liquid formulations of the current invention.

In an embodiment, the soft mist inhalation devices suitable for administering the pharmaceutical formulations of the present invention can nebulize less than about 30 microliters of biopharmaceutical solution to produce an aerosol delivering a therapeutically effective quantity of the drug. An average particle size of aerosol formed from one puff is less than about 10 micrometer, such as less than about 5 micrometer. The biopharmaceutical formulation in the soft mist inhaler is converted into aerosol destined for lung deposition. The pharmaceutical formulation is stored in a reservoir in the soft mist inhaler. In an embodiment, the pharmaceutical formulation does not contain any ingredients which might interact with the inhaler to affect the quality of the solution or of the aerosol produced. In addition, in an embodiment, the active substances in the biopharmaceutical formulations according to the present invention are very stable when stored at 2° C. to 8° C. and can be administered directly.

In one embodiment of the current invention, the inhalation device can be a soft mist inhaler. In an embodiment, to produce the aerosols according to the invention, the pharmaceutical soft mist bio-formulations containing a therapeutic monoclonal antibody is used in an inhaler of the kind described herein. Here we should once again briefly mention the patent documents described hereinbefore, to which reference is hereby made.

A soft mist inhaler device of this kind for the propellant-free administration of a metered amount of a liquid biopharmaceutical composition for soft mist inhalation is described in detail, for example, in US20190030268 “inhalation atomizer comprising a blocking function and a counter”.

The biopharmaceutical formulation in the nebulizer is converted into aerosol destined for the lungs. The biopharmaceutical solution is sprayed by the nebulizer using high pressure.

The soft mist inhalation device can be carried anywhere by the patient, since it has a cylindrical shape and a handy size of less than about 8 cm to 18 cm long and 2.5 cm to 5 cm wide. The nebulizer sprays a defined volume of the pharmaceutical formulation out through small nozzles at high pressures, so as to produce inhalable aerosols.

The preferred atomizer comprises an atomizer 1, a fluid 2, a vessel 3, a fluid compartment 4, a pressure generator 5, a holder 6, a drive spring 7, a delivering tube 9, a non-return valve 10, pressure room 11, a nozzle 12, a mouthpiece 13, an aerosol 14, an air inlet 15, an upper shell 16, and an inside part 17.

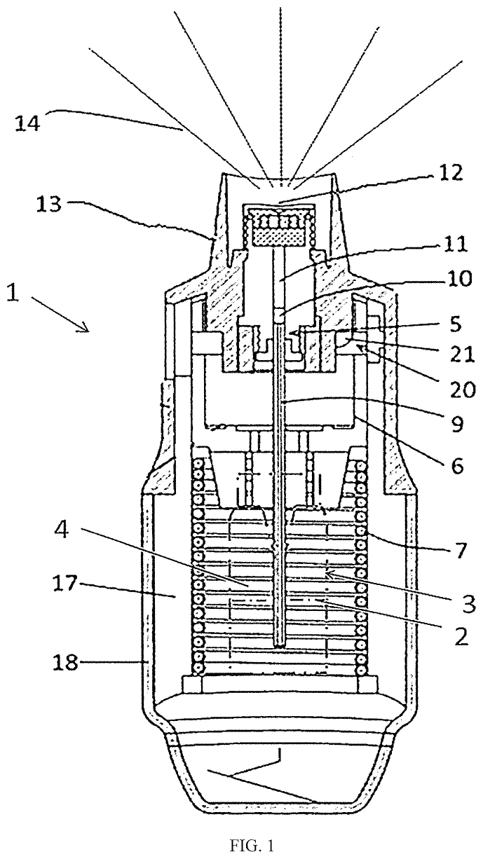

The inhalation atomizer 1 comprising the block function and the counter described above for spraying a medicament fluid 2 is depicted in FIG. 1 in a stressed state. The atomizer 1 comprising the block function and the counter described above is preferred as a portable inhaler and requires no propellant gas.

FIG. 1 shows a longitudinal section through the atomizer in the stressed state. For the typical atomizer 1 comprising the block function and the counter described above, an aerosol 14 that can be inhaled by a patient is generated through the atomization of the fluid 2, which is preferably formulated as a medicament liquid. The medicament is typically administered at least once a day, more specifically multiple times a day, preferably at predetermined time gaps, according to how seriously the illness affects the patient.

In an embodiment, the atomizer 1 described above has substitutable and insertable vessel 3, which contains the medicament fluid 2. A reservoir for holding the fluid 2 is formed in the vessel 3. Specifically, the medicament fluid 2 is located in the fluid compartment 4 formed by a collapsible bag in the vessel 3.

In an embodiment, the amount of fluid 2 for the inhalation atomizer 1 described above is in the vessel 3 to provide, e.g., up to 200 doses. A typical vessel 3 has a volume of about 2 ml to about 10 ml. A pressure generator 5 in the atomizer 1 is used to deliver and atomize the fluid 2 in a predetermined dosage amount. The fluid 2 can be released and sprayed in individual doses, specifically from about 5 to about 30 microliters.

In an embodiment, the atomizer 1 described above may have a pressure generator 5 and a holder 6, a drive spring 7, a delivering tube 9, a non-return valve 10, a pressure room 11, and a nozzle 12 in the area of a mouthpiece 13. The vessel 3 is latched by the holder 6 in the atomizer 1 so that the delivering tube 9 is plunged into the vessel 3. The vessel 3 can be separated from the atomizer 1 for substitution.

In an embodiment, when drive spring 7 is stressed in an axial direction, the delivering tube 9, the vessel 3 along with the holder 6 will be shifted downwards. Then the fluid 2 will be sucked into the pressure room 11 through delivering tube 9 and the non-return valve 10.

In one embodiment, after releasing the holder 6, the stress is eased. During this process, the delivering tube 9 and closed non-return valve 10 are shifted back upward by releasing the drive spring 7. Consequently, the fluid 2 is under pressure in the pressure room 11. The fluid 2 is then pushed through the nozzle 12 and atomized into an aerosol 14 by the pressure. A patient can inhale the aerosol 14 through the mouthpiece 13, while the air is sucked into the mouthpiece 13 through air inlets 15.

The inhalation atomizer 1 described above has an upper shell 16 and an inside part 17, which can be rotated relative to the upper shell 16. A lower shell 18 is manually operable to attach onto the inside part 17. The lower shell 18 can be separated from the atomizer 1 so that the vessel 3 can be substituted and inserted.

In one embodiment of the inhalation atomizer 1 described above has a lower shell 18, which carries the inside part 17, and is rotatable relative to the upper shell 16. As a result of rotation and engagement between the upper unit 17 and the holder 6, through a gear 20, the holder 6 is axially moved counter to the force of the drive spring 7, and the drive spring 7 is stressed.

In an embodiment, in the stressed state, the vessel 3 is shifted downwards and reaches a final position, which is demonstrated in FIG. 1. The drive spring 7 is stressed in this final position. Then the holder 6 is clasped. Therefore, the vessel 3 and the delivering tube 9 are prevented from moving upwards so that the drive spring 7 is stopped from easing.

In an embodiment, the atomizing process occurs after releasing the holder 6. The vessel 3, the delivering tube 9 and the holder 6 are shifted back by the drive spring 7 to the beginning position. This is referred to herein as major shifting. When major shifting occurs, the non-return valve 10 is closed and the fluid 2 is under pressure in the pressure room 11 by the delivering tube 9, and then the fluid 2 is pushed out and atomized by the pressure.

In an embodiment, the inhalation atomizer 1 described above may have a clamping function. During the clamping, the vessel 3 preferably performs a lifting shift for the withdrawal of fluid 2 during the atomizing process. The gear 20 has sliding surfaces 21 on the upper shell 16 and/or on the holder 6, which can make holder 6 move axially when the holder 6 is rotated relative to the upper shell 16.

In an embodiment, the holder 6 is not blocked for too long and can perform the major shifting. Therefore, the fluid 2 is pushed out and atomized. In an embodiment, when holder 6 is in the clamping position, the sliding surfaces 21 move out of engagement. Then the gear 20 releases the holder 6 for the opposite axial shift.

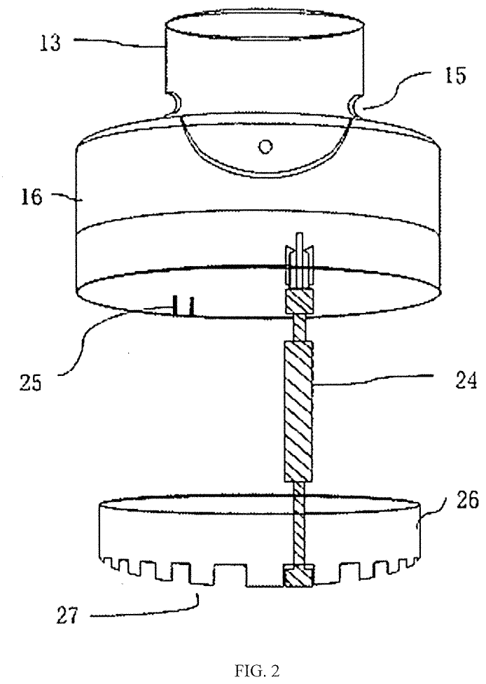

In one embodiment, the atomizer 1 includes a counter element as shown in FIG. 2. The counter element has a worm 24 and a counter ring 26. The counter ring 26 is preferably circular and has dentate part at the bottom. The worm 24 has upper and lower end gears. The upper end gear contacts with the upper shell 16. The upper shell 16 has inside bulge 25. When the atomizer 1 is employed, the upper shell 16 rotates; and when the bulge 25 passes through the upper end gear of the worm 24, the worm 24 is driven to rotate. The rotation of the worm 24 drives the rotation of the counter ring 26 through the lower end gear so as to result in a counting effect.

In an embodiment, the locking mechanism is realized mainly by two protrusions. Protrusion A is located on the outer wall of the lower unit of the inside part. Protrusion B is located on the inner wall of counter. The lower unit of the inside part is nested in the counter. The counter can rotate relative to the lower unit of the inside part. Because of the rotation of the counter, the number displayed on the counter can change as the actuation number increases, and can be observed by the patient. After each actuation, the number displayed on the counter changes. Once the predetermined number of actuations is achieved, Protrusion A and Protrusion B will encounter each other and the counter will be prevented from further rotation. Therefore, the atomizer is blocked and stopped from further use. The number of actuations of the device can be counted by the counter.

The nebulizer described above is suitable for nebulizing the pharmaceutical formulations of the invention to form an aerosol suitable for inhalation. Nevertheless, the formulation according to the invention can also be nebulized using other inhalers apart from those described above, such as ultrasonic nebulizers, jet nebulizers or mesh nebulizers.

The effectiveness of a soft mist inhaler can be tested using an in vitro system in which a therapeutic monoclonal antibody solution is aerosolized and the soft mist is caught in a so-called ‘trap’. The activity of the therapeutic antibody in the aerosol reservoir (a) can be compared with its activity in the trapped liquid (b), e.g. by means of an immunoassay or using an assay for the biological effectiveness of the antibody. This experiment makes it possible to evaluate the degree of destruction of the functional antibody drug by the nebulizing process. A second parameter of the aerosol quality is the so-called inhalable proportion, which is defined here as the proportion of the mist droplets with a measured mass median aerodynamic diameter (MMAD) of less than 10 micrometer. The inhalable proportion can be measured using an “Andersen Impactor”. For good protein inhalation and absorption it is important not only to achieve aerosolization without any substantial loss of activity but also to generate an aerosol with a good inhalable proportion. Aerosols with an MMAD of less than 5 micrometer are significantly better suited to reaching the alveoli, where their chances of being absorbed are significantly greater. The effectiveness of a soft mist inhalation device can also be tested in an in vivo system; in this case factors such as susceptibility to lung proteases come into play. As an example of an in vivo test system, a protein containing mist can be administered to a dog through a tracheal tube. Blood samples are taken at suitable time intervals and the protein level in the plasma is then measured by immunological or biological methods.

Another advantage of the invention claimed is its surprising ability to nebulize optimum concentrated solutions of biologically active macromolecules without any substantial loss of activity.

### EXAMPLES

Formulation Ingredients:
Programmed cell death receptor-1 (PD-1) blocking antibody—pembrolizumab, nivolumab,
Programmed cell death ligand-1 (PD-L1) blocking antibody—atezolizumab, durvalumab
Vascular endothelial growth factor receptor blocking antibody—bevacizumab
Polysorbate 80
Polysorbate 20
L-histidine
L-histidine hydrochloride monohydrate
Sodium citrate dihydrate
Sodium chloride
Sodium Phosphate (monobasic, monohydrate)
Sodium phosphate (dibasic, dihydrate)
Mannitol
Sucrose
Glacial acetic acid
Pentetic acid

### Example 1

A formulation of an aqueous solution containing a programmed cell death receptor-1 blocking therapeutic antibody as an active ingredient for soft mist inhaler and/or nebulizer was mixed and formulated with excipients listed in Table 1. Stock solutions of excipients are prepared in 10-50 times higher concentration prior to preparing the bio-formulation with the therapeutic monoclonal antibody to bring it to the desired concentration.

TABLE 1
Formulation ingredient contents of Sample I and Sample II

| Ingredients | Sample I | | Sample II | |
|---|---|---|---|---|
| Programmed cell death receptor-1 | 50 | mg | 100 | mg |
| (PD-1) blocking antibody | | | | |
| (Pembrolizumab) | | | | |
| L-histidine | 3.1 | mg | 6.2 | mg |
| Polysorbate 80 | 0.4 | mg | 0.8 | mg |
| Sucrose | 140 | mg | 280 | mg |
| pH | 5.5 | | 5.5 | |
| Water for injection | 5 | ml | 5 | ml |

### Example 2

A formulation of an aqueous solution containing a programmed cell death ligand-1 blocking therapeutic antibody as an active ingredient for soft mist inhaler and/or nebulizer was mixed and formulated with excipients listed in Table 2. Stock solutions of excipients are prepared in 10-50 times higher concentration prior to preparing the bio-formulation with the therapeutic monoclonal antibody to bring it to the desired concentration.

TABLE 2
Formulation ingredient contents of Sample III, Sample IV
and Sample V

| Ingredients | Sample III | | Sample IV | | Sample V | |
|---|---|---|---|---|---|---|
| Programmed cell death ligand-1 | 75 | mg | 150 | mg | 300 | mg |
| (PD-L1) blocking antibody | | | | | | |
| (Atezolizumab) | | | | | | |
| L-histidine | 77.5 | mg | 155 | mg | 310 | mg |
| Polysorbate 20 | 10 | mg | 20 | mg | 40 | mg |
| Sucrose | 1027 | mg | 2054 | mg | 4108 | mg |
| Glacial acetic acid | 20.625 | mg | 41.25 | mg | 82.8 | mg |
| pH | 5.8 | | 5.8 | | 5.8 | |
| Water for injection | 5 | ml | 5 | ml | 5 | ml |

### Example 3

A formulation of an aqueous solution containing a programmed cell death receptor-1 blocking therapeutic antibody as an active ingredient for soft mist inhaler and/or nebulizer was mixed and formulated with excipients listed in Table 3. Stock solutions of excipients are prepared in 10-50 times higher concentration prior to preparing the bio-formulation with the therapeutic monoclonal antibody to bring it to the desired concentration.

TABLE 3
Formulation ingredient contents of Sample VI and Sample VII

| Ingredients | Sample VI | | Sample VII | |
|---|---|---|---|---|
| Programmed cell death receptor-1 | 25 | mg | 50 | mg |
| (PD-1) blocking antibody | | | | |
| (Nivolumab) | | | | |
| Mannitol | 75 | mg | 150 | mg |
| Sodium chloride | 7.3 | mg | 14.6 | mg |
| Polysorbate 80 | 0.5 | mg | 1 | mg |
| Sodium citrate dihydrate | 14.7 | mg | 29.4 | mg |
| Pentetic acid | 0.02 | mg | 0.04 | mg |
| pH | 6 | | 6 | |
| Water for injection USP | 5 | ml | 5 | ml |

### Example 4

A formulation of an aqueous solution containing a programmed cell death receptor-1 blocking therapeutic antibody as an active ingredient for soft mist inhaler and/or nebulizer was mixed and formulated with excipients listed in Table 4. Stock solutions of excipients are prepared in 10-50 times higher concentration prior to preparing the bio-formulation with the therapeutic monoclonal antibody to bring it to the desired concentration.

TABLE 4
Formulation ingredient contents of Sample VIII, Sample IX and
Sample X

| Ingredients | Sample VIII | | Sample IX | | Sample X | |
|---|---|---|---|---|---|---|
| Programmed cell death ligand-1 | 62.5 | mg | 125 | mg | 250 | mg |
| (PD-L1) blocking antibody | | | | | | |
| (Durvalumab) | | | | | | |
| L-histidine | 2.5 | mg | 5 | mg | 10 | mg |
| L-histidine hydrochloride | 3.375 | mg | 6.75 | mg | 13.5 | mg |
| monohydrate | | | | | | |
| α,α,-trehalose dihydrate | 130 | mg | 260 | mg | 520 | mg |
| Polysorbate 80 | 0.25 | mg | 0.5 | mg | 1 | mg |
| pH | 6 | | 6 | | 6 | |
| Water for injection | 5 | ml | 5 | ml | 5 | ml |

### Example 5

A formulation of an aqueous solution containing a programmed cell death receptor-1 blocking therapeutic antibody as an active ingredient for soft mist inhaler and/or nebulizer was mixed and formulated with excipients listed in Table 5. Stock solutions of excipients are prepared in 10-50 times higher concentration prior to preparing the bio-formulation with the therapeutic monoclonal antibody to bring it to the desired concentration.

TABLE 5
Formulation ingredient contents of Sample XI, Sample XII and
Sample XIII

| | Sample | | Sample | | Sample | |
|---|---|---|---|---|---|---|
| Ingredients | XI | | XII | | XIII | |
| Vascular endothelial growth | 50 | mg | 75 | mg | 125 | mg |
| factor blocking antibody | | | | | | |
| (Bevacizumab) | | | | | | |
| α,α-trehalose dihydrate | 120 | mg | 180 | mg | 300 | mg |
| Sodium phosphate | 11.6 | mg | 17.4 | mg | 29 | mg |
| (monobasic, monohydrate) | | | | | | |
| Sodium phosphate | 2.4 | mg | 3.6 | mg | 6 | mg |
| (dibasic, anhydrous) | | | | | | |
| Polysorbate 20 | 0.8 | mg | 1.2 | mg | 2 | mg |
| pH | 5.8 | | 5.8 | | 5.8 | |
| Water for injection | 5 | ml | 5 | ml | 5 | ml |

### Example 6

Aerodynamic Particle Size Distribution:

The aerodynamic particle size distribution was determined using a Next Generation Pharmaceutical Impactor (NGI). The sample is Sample I in Example 1.

The device is a soft mist inhaler, as disclosed in US20190030268, “inhalation atomizer comprising a blocking function and a counter”.

The device was held close to the NGI inlet until no aerosol was visible. The flow rate of the NGI was set to 30 L/minute and was operated under ambient temperature and a relative humidity (RH) of 90%.

The solution of Sample I in Example 1 was discharged into the NGI. Fractions of the dose were deposited at different stages of the NGI, in accordance with the particle size of the fraction. Each fraction was washed from the stage and analyzed using HPLC.

The result is shown in Table 6.

TABLE 6
Aerodynamic particle size distribution of Sample I in Example 1

| | | | Cut-off |
|---|---|---|---|
| | pembrolizumab | | diameters |
| | | Percentage | at |
| | Dosage | content at all | 30 L/min |
| Deposited | (μg) | levels % | (μm) |
| Throat | 33.71 | 15.66 | |
| Stage 1 | 11.40 | 5.30 | 11.72 |
| Stage 2 | 32.69 | 15.19 | 6.4 |
| Stage 3 | 54.03 | 25.11 | 3.99 |
| Stage 4 | 50.65 | 23.54 | 2.30 |
| Stage 5 | 22.66 | 10.53 | 1.36 |
| Stage 6 | 5.62 | 2.61 | 0.83 |
| Stage 7 | 1.07 | 0.50 | 0.54 |
| MOC | 3.34 | 1.55 | |

| | |
|---|---|
| Theoretical dose (μg) | 221 |
| Actual test dose (μg) | 215.17 |
| Recovery rate (%) | 97.36 |
| ISM(μg) | 170.07 |
| FPD(μg) | 137.37 |
| FPF(%) | 63.85 |

MOC is Micro-Orifice Collector.
ISM is Impactor Size Mass.
FPF is Fine Particle Fraction.
FPD is fine particle dose.

### Example 7

Aerodynamic Particle Size Distribution:

The aerodynamic particle size distribution was determined using a Next Generation Pharmaceutical Impactor (NGI). The sample is Sample IX in Example 4.

The device is a soft mist inhaler, as disclosed in US20190030268, “inhalation atomizer comprising a blocking function and a counter”.

The device was held close to the NGI inlet until no aerosol was visible. The flow rate of the NGI was set to 30 L/minute and was operated under ambient temperature and a relative humidity (RH) of 90%.

The solution of Sample IX in Example 4 was discharged into the NGI. Fractions of the dose were deposited at different stages of the NGI, in accordance with the particle size of the fraction. Each fraction was washed from the stage and analyzed using HPLC.

The result is shown in Table 7.

TABLE 7
Aerodynamic particle size distribution of Sample IX in Example 4

| | | | Cut-off |
|---|---|---|---|
| | Durvalumab | | diameters |
| | | Percentage | at |
| | Dosage | content at | 30 L/min |
| Deposited | (μg) | all levels % | (μm) |
| Throat | 112.15 | 19.55 | |
| Stage 1 | 33.08 | 5.77 | 11.72 |
| Stage 2 | 82.19 | 14.33 | 6.4 |
| Stage 3 | 150.03 | 26.15 | 3.99 |
| Stage 4 | 125.05 | 21.80 | 2.30 |
| Stage 5 | 46.13 | 8.04 | 1.36 |
| Stage 6 | 13.25 | 2.31 | 0.83 |
| Stage 7 | 3.15 | 0.55 | 0.54 |
| MOC | 8.63 | 1.50 | |

| | |
|---|---|
| Theoretical dose (μg) | 552.5 |
| Actual test dose (μg) | 573.66 |
| Recovery rate (%) | 103.83 |
| ISM(μg) | 428.43 |
| FPD(μg) | 346.24 |
| FPF(%) | 60.36 |

### Example 8

Aerodynamic Particle Size Distribution:

The aerodynamic particle size distribution was determined using a Next Generation Pharmaceutical Impactor (NGI). The sample is Sample XI in Example 5.

The device is a soft mist inhaler, as disclosed in US20190030268, “inhalation atomizer comprising a blocking function and a counter”.

The device was held close to the NGI inlet until no aerosol was visible. The flow rate of the NGI was set to 30 L/minute and was operated under ambient temperature and a relative humidity (RH) of 90%.

The solution of Sample XI in Example 5 was discharged into the NGI. Fractions of the dose were deposited at different stages of the NGI, in accordance with the particle size of the fraction. Each fraction was washed from the stage and analyzed using HPLC.

The result is shown in Table 8.

TABLE 8
Aerodynamic particle size distribution of Sample XI in Example 5

| | | | Cut-off |
|---|---|---|---|
| | Bevacizumab | | diameters |
| | | Percentage | at |
| | Dosage | content at | 30 L/min |
| Deposited | (μg) | all levels % | (μm) |
| Throat | 56.18 | 17.64 | |
| Stage 1 | 18.02 | 5.66 | 11.72 |
| Stage 2 | 46.07 | 14.47 | 6.4 |
| Stage 3 | 77.54 | 24.35 | 3.99 |
| Stage 4 | 72.36 | 22.72 | 2.30 |
| Stage 5 | 32.01 | 10.05 | 1.36 |
| Stage 6 | 9.30 | 2.92 | 0.83 |
| Stage 7 | 1.66 | 0.52 | 0.54 |
| MOC | 5.32 | 1.67 | |

| | |
|---|---|
| Theoretical dose (μg) | 331.5 |
| Actual test dose (μg) | 318.45 |
| Recovery rate (%) | 96.06 |
| ISM(μg) | 244.25 |
| FPD(μg) | 198.19 |
| FPF(%) | 62.24 |

### Example 9

Stability Studies:

A formulation of an aqueous solution containing a programmed cell death receptor-1 blocking therapeutic antibody as an active ingredient for administration using a soft mist inhaler and/or nebulizer was mixed and formulated with excipients listed in Table 9. Stock solutions of excipients are prepared in 10-50 times higher concentration prior to preparing the formulation with the therapeutic monoclonal antibody and are diluted to the desired concentration.

TABLE 9
Formulation Ingredients of Sample XIV

| Ingredients | Sample XIV | |
|---|---|---|
| Vascular endothelial growth factor | 75 | mg |
| blocking antibody | | |
| (Bevacizumab) | | |
| α,α-trehalose dihydrate | 180 | mg |
| Sodium phosphate | 17.4 | mg |
| (monobasic, monohydrate) | | |
| Sodium phosphate | 3.6 | mg |
| (dibasic, anhydrous) | | |
| Polysorbate 20 | 1.2 | mg |
| pH | 6.75 | |
| Water for injection | 5 | ml |

TABLE 10
Accelerated and Long Term Stability

| | | Accelerated Storage Condition | | | |
|---|---|---|---|---|---|
| | | (25° C. ± 2° C./60% RH ± 5% RH) | | | |
| Content | Initial | 1 month | 2 months | 3 months | 6 months |
| Description | Clear | Clear | Clear | Clear | Clear |
| | colorless | colorless | colorless | colorless | colorless |
| | Solution | Solution | Solution | Solution | Solution |
| Bevacizumab | 102.0 | 102.5 | 101.9 | 102.5 | 100.9 |
| Content | | | | | |
| (% w/w) | | | | | |
| pH | 6.75 | 6.78 | 6.81 | 6.83 | 6.80 |
| Molecular | Complies | Complies | Complies | Complies | Complies |
| weight | | | | | |
| High | Complies | Complies | Complies | Complies | Complies |
| molecular | | | | | |
| weight | | | | | |
| impurities | | | | | |
| Low | Complies | Complies | Complies | Complies | Complies |
| molecular | | | | | |
| weight | | | | | |
| impurities | | | | | |

TABLE 11
Accelerated and Long Term Stability

| | | Long Term Storage Condition | |
|---|---|---|---|
| | | (2-8° C./25% RH ± 5% RH) | |
| Content | Initial | 3 months | 6 months |
| Description | Clear | Clear to | Clear to |
| | colorless | opalescent, | opalescent, |
| | Solution | colorless to | colorless to |
| | | pale yellow | pale yellow |
| Bevacizumab | 102.0 | 102.1 | 102.0 |
| Content (% w/w) | | | |
| pH | 6.75 | 6.77 | 6.73 |
| Molecular weight | Complies | Complies | Complies |
| Free thiols | Complies | Complies | Complies |
| High molecular | Complies | Complies | Complies |
| weight impurities | | | |
| Low molecular | Complies | Complies | Complies |
| weight impurities | | | |

The experimental results show that the bevacizumab-containing formulation has good stability.

What is claimed is:

1. A pharmaceutical formulation suitable for administration by soft mist inhalation or nebulization comprising:
(a) a therapeutic monoclonal antibody in a concentration of from about 1 mg/ml to about 100 mg/ml,
(b) water, and
(c) a buffer,
wherein the therapeutic monoclonal antibody is selected from the group consisting of pembrolizumab, atezolizumab, nivolumab, durvalumab, and bevacizumab.

2. A method of administering the pharmaceutical formulation of claim 1 comprising inhalation via an inhalation device selected from a soft mist inhalation device and a nebulization device.

3. The pharmaceutical formulation of claim 1 wherein
the therapeutic monoclonal antibody is pembrolizumab in a concentration of from about 1 mg/ml to about 25 mg/ml;
the buffer comprises from about 1 mM to about 10 mM of L-histidine, from about 50 mM to about 200 mM sucrose, and from about 0.04 mM to about 0.15 mM polysorbate 80;
the pharmaceutical formulation has a pH of from about 5.5 to about 5.8; and
the pharmaceutical formulation is stable for at least about 12 months when stored under refrigerated conditions at about 2° C. to about 8° C.

4. The pharmaceutical formulation of claim 1 wherein
the therapeutic monoclonal antibody is atezolizumab in an amount of from about 10 mg/ml to about 65 mg/ml;
the buffer comprises from about 50 mM to about 450 mM of L-histidine, from about 500 mM to about 2450 mM sucrose, from about 1 mM to about 10 mM polysorbate 20, and from about 60 mM to about 300 mM of glacial acetic acid;
the pharmaceutical formulation has a pH of from about 5.8 to about 6; and
the pharmaceutical formulation is stable for at least about 12 months when stored under refrigerated conditions at about 2° C. to about 8° C.

5. The pharmaceutical formulation of claim 1 wherein
the therapeutic monoclonal antibody is nivolumab in an amount of from about 5 mg/ml to about 10 mg/ml;
the buffer comprises from about 0.05 mM to about 200 mM of polysorbate 80, from about 10 mM to about 30 mM sodium citrate dihydrate, from about 20 mM to about 60 sodium chloride, and from about 50 mM to about 200 mM of mannitol, and from about 0.005 mM to about 0.025 mM pentetic acid;
the pharmaceutical formulation has a pH of about 6; and
the pharmaceutical formulation is stable for at least about 12 months when stored under refrigerated conditions at about 2° C. to about 8° C.

6. The pharmaceutical formulation of claim 1 wherein
the therapeutic monoclonal antibody is durvalumab in an amount of from about 10 mg/ml to about 55 mg/ml;
the buffer comprises from about 1 mM to about 15 mM of L-histidine, from about 0.03 mM to about 0.2 mM polysorbate 80, from about 1 mM to about 15 L-histidine hydrochloride monohydrate, and from about 50 mM to about 300 mM of trehalose dihydrate;
the pharmaceutical formulation has a pH of about 6; and
the pharmaceutical formulation is stable for at least about 12 months when stored under refrigerated conditions at about 2° C. to about 8° C.

7. The pharmaceutical formulation of claim 1 wherein
the therapeutic monoclonal antibody is bevacizumab in an amount of from about 10 mg/ml to about 25 mg/ml;
the buffer comprises from about 0.15 mM to about 0.35 mM polysorbate 20, from about 15 mM to about 55 monobasic sodium phosphate monohydrate, and from about 1 mM to about 10 mM of dibasic sodium phosphate dihydrate, and from about 50 mM to about 200 mM of trehalose dehydrate;
the pharmaceutical formulation has a pH of about 6; and
the pharmaceutical formulation is stable for at least about 12 months when stored under refrigerated conditions at about 2° C. to about 8° C.

8. A method of administering the pharmaceutical formulation of claim 3 comprising producing an aerosol using an inhalation device selected from a soft mist inhalation device and a nebulization device.

9. A method of administering the pharmaceutical formulation of claim 4 comprising producing an aerosol using an inhalation device selected from a soft mist inhalation device and a nebulization device.

10. A method of administering the pharmaceutical formulation of claim 5 comprising producing an aerosol using an inhalation device selected from a soft mist inhalation device and a nebulization device.

11. A method of administering the pharmaceutical formulation of claim 6 comprising producing an aerosol using an inhalation device selected from a soft mist inhalation device and a nebulization device.

12. A method of administering the pharmaceutical formulation of claim 7 comprising producing an aerosol using an inhalation device selected from a soft mist inhalation device and a nebulization device.

13. A method for treating lung cancer comprising administering the formulation of claim 1 to a patient by inhalation.

14. A method for treating lung cancer comprising administering the formulation of claim 3 to a patient by inhalation.

15. A method for treating lung cancer comprising administering the formulation of claim 4 to a patient by inhalation.

16. A method for treating lung cancer comprising administering the formulation of claim 5 to a patient by inhalation.

17. A method for treating lung cancer comprising administering the formulation of claim 6 to a patient by inhalation.

18. A method for treating lung cancer comprising administering the formulation of claim 7 to a patient by inhalation.

19. The method of claim 2, wherein the therapeutic monoclonal antibody is administered in a daily dose ranging from about 1 μg to about 2000 mg per dose.
